(19) United States
(12) Patent Application Publication
(10) Pub. No.: US 2021/0304879 A1
(43) Pub. Date: Sep. 30, 2021

(54) METHODS, SYSTEMS, AND COMPUTER PROGRAM PRODUCTS FOR DIVIDING HEALTH CARE SERVICE RESPONSIBILITIES BETWEEN ENTITIES

(71) Applicant: Change Healthcare Holdings LLC, Nashville, TN (US)

(72) Inventors: Scott Morris, Greenville, SC (US); Faisal Mushtaq, West Harrison, NY (US); Jody Terakawa, Brentwood, TN (US)

(21) Appl. No.: 17/213,858

(22) Filed: Mar. 26, 2021

Related U.S. Application Data
(60) Provisional application No. 63/002,749, filed on Mar. 31, 2020.

(51) Int. Cl. G16H 40/20; G16H 10/60; G16H 50/20; G06Q 30/04; G06Q 40/08; G16H 10/20

(52) U.S. Cl. CPC G16H 40/20; G16H 10/60; G16H 10/20; G06Q 30/04; G06Q 40/08; G16H 50/20

(57) ABSTRACT

A method includes receiving information associated with health care services provided to a patient; determining for respective ones of the health care services respective probabilities that a first entity will refuse responsibility; associating a first portion of the health care services with the first entity; associating a second portion of the health care services with a second entity, the second portion of the health care services comprising respective ones of the health care services having probabilities that exceed a defined threshold; assigning responsibility for the first portion of the health care services to the first entity; and assigning responsibility for the second portion of the health care services to the second entity.

### CROSS-REFERENCE TO RELATED APPLICATION

This application claims the benefit of priority to U.S. Provisional Patent Application No. 63/002,749 entitled “Methods, Systems, and Computer Program Products for Dividing Health Care Service Responsibilities Between Entities,” filed on Mar. 31, 2020, in the United States Patent and Trademark Office, the disclosure of which is incorporated by reference herein.

### FIELD

The present inventive concepts relate generally to health care systems and services and, more particularly, management of health care service responsibilities among multiple entities.

### BACKGROUND

Clinical trials are research studies performed on subjects (patients) that are aimed at evaluating a medical, surgical, or behavioral intervention. They are a commonly used way that researchers find out if a new treatment, such as a new drug, treatment, or medical device, is safe and effective in people. A clinical trial may often be used to learn if a new treatment is more effective and/or has less harmful side effects than a standard treatment. It is difficult, however, for trial sponsors to recruit physicians and other health care personnel to participate in trials. One reason that health care service providers have identified as discouraging them from trial participation is the additional administrative burden and overhead expense for the people and systems used to support billing a trial sponsor for the clinical trial services performed and reconciling payments associated with those studies. To participate in a clinical trial, a medical facility or practice may be required to establish a separate system for capturing records associated with the health care services (i.e., clinical data submission) along with a separate invoicing system for allocating the cost of the services connected to participating in the trial to the trial sponsor. This duplicative system result is sometimes referred to as a two-rail system, which is illustrated in FIG. 1. As shown in FIG. 1, a health care service provider 102 may be recruited to participate in a clinical trial. Medical records for patients enrolled in the trial may be processed using the clinical trial information module 105 to extract clinical data therefrom for the services that are relevant to the trial or these data are entered manually into a clinical trial data submission system. These medical records may then be processed by a clinical trial management system 110 to create invoices for the expenses and fees generated by the provider 102 that are associated with the clinical trial. These invoices may then be submitted to the trial sponsor 115 or a company operating on their behalf, which may be referred to as a Contract Research Organization (CRO), for payment. The CRO/trial sponsor 115 may process the invoices and remit payment to the provider 102. A trial reconciliation system 118 may be used to reconcile the payments made by the CRO/trial sponsor 115 with the invoices that were issued, which may include tracking any invoices that are still pending payment and tracking any invoices that were rejected or declined in whole or in part to alert the provider 102 that the original invoice may need to be revisited.

The clinical trial process flow has a counterpart health care treatment process flow for health care services provided to a patient as part of routine care. The routine care includes services associated with standard of care treatment, which is used to denote those services that a payor, such as an insurer, should be responsible for. In the health care treatment process flow, the provider 102 may provide health care services to patients as part of their routine care. The medical records for these patients may be processed using the health care service information module 120 to extract data therefrom documenting the various services and products that the patients have received. This data is then processed by a medical billing network 130 to create invoices for the expenses and fees generated by the provider 102 that are associated with delivery of health care treatment services. These invoices may then be submitted to a payor, such as an insurer 135 (e.g., private insurer, government insurer (Medicare, Medicaid), etc.), for payment. The insurer 135 may process the invoices and remit payment to the provider 102. Similar to the trial reconciliation system 118, a health care treatment reconciliation system 138 may be used to reconcile the payments made by the insurer 135 with the invoices that were issued, which may include tracking any invoices that are still pending payment and tracking any invoices that were rejected or declined in whole or in part to alert the provider 102 that the original invoice may need to be revisited.

Thus, when a provider 102 agrees to participate in a clinical trial, the provider 102 along with the health care facility where the provider practices are required to establish these two process flows in which clinical trial data, invoices for CROs/sponsors 115, and reconciliation of remittances from CROs/sponsors 115 are kept separate from health care treatment data, invoices for insurers 135, and reconciliation of remittances from insurers 135. In addition to the overhead, providers 102 may have to make difficult decisions in determining whether certain health care services are part of routine health care treatment or are only connected to the clinical trial. Providers may be penalized or held criminally liable if they invoice insurers 135, particularly government insurers like Medicare, for all medical services and only invoice a CRO/trial sponsor 115 for invoices that are rejected or declined by the insurer 135. CROs/sponsors 115, however, may be overbilled, in some instances, if providers 102 err on the side of billing CROs/sponsors 115 for most services when some or many of these services may have been covered under health care treatment benefit plans for the patients. Because of the uncertainty in what portion of the health care services delivered to patients that are participating in trials will ultimately be allocated to them, CROs/sponsors 115 may carry large accrual balances to ensure they have sufficient funds available for unexpected invoices associated with a trial.

The parallel systems used to manage clinical trials and health care treatment services may create risks for providers 102, CROs/sponsors 115, and insurers 135 due to difficulties in accurately dividing responsibilities for the health care services between the CROs/sponsors 115 and the insurers 135. As a result, it is estimated that less than 10% of physicians agree to participate in clinical trials, which may hamper the ability to make clinical trials more widely accessible to patients, which may also hamper the ability to get new drugs, medical treatments, and the like approved.

### SUMMARY

According to some embodiments of the inventive concept, a method comprises receiving information associated with health care services provided to a patient; determining for respective ones of the health care services respective probabilities that a first entity will refuse responsibility; associating a first portion of the health care services with the first entity; associating a second portion of the health care services with a second entity, the second portion of the health care services comprising respective ones of the health care services having probabilities that exceed a defined threshold; assigning responsibility for the first portion of the health care services to the first entity; and assigning responsibility for the second portion of the health care services to the second entity.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims. It is further intended that all embodiments disclosed herein can be implemented separately or combined in any way and/or combination.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram of a two-rail system for managing health care treatment services and clinical trial services;

FIG. 2 is a block diagram that illustrates a communication network including a multiple entity management system for dividing health care service responsibilities between entities in accordance with some embodiments of the inventive concept;

FIGS. 3-8 are flowcharts that that illustrate operations for dividing health care service responsibilities between entities in accordance with some embodiments of the inventive concept;

FIG. 9 is a message flow diagram that illustrates operations for dividing health care service responsibilities between entities in accordance with some embodiments of the inventive concept;

FIG. 10 is a data processing system that may be used to implement one or more servers in the multiple entity management system of FIG. 2 in accordance with some embodiments of the inventive concept; and

FIG. 11 is a block diagram that illustrates a software/hardware architecture for use in the multiple entity management system of FIG. 2 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care services to a patient. When a provider participates in a clinical trial, the provider may be called an “investigator.” When a patient participates in a clinical trial, the patient may be called a “subject.”

Some embodiments of the inventive concept stem from a realization that maintaining two independent systems for managing patient care when a patient is enrolled in a clinical trial may increase the administrative overhead involved for providers, medical centers, medical practices, etc. who participate in clinical trials. In addition to the added administrative burden, the difficulties in accurately allocating responsibility for medical services provided to a patient who is enrolled in a clinical trial between a Contract Research Organization (CRO)/trial sponsor payor and an insurance payor may place a provider in jeopardy of being fined or held criminally liable if an insurance payor is overbilled. To avoid such risks, providers may bill CROs/trial sponsors more than what they should be responsible for based on, for example, a patient's health care treatment services benefits under an insurance policy. The administrative burden in setting up parallel systems for managing clinical trial services and health care treatment services along with the risks that accompany inaccurately dividing responsibility for patient care between a CRO/trial sponsor responsible for the clinical trial services and an insurer responsible for the health care treatment services may deter providers, medical centers, and/or medical practices from participating in clinical trials, which may be an important part of bringing new drugs, medical devices, and the like to market.

According to some embodiments of the inventive concept, a single multiple entity management unified system may be provided that can process data associated with a patient's treatment and associate the data with different entities. For example, a trial sponsor or CRO may be collecting data on services rendered that are relevant to the trial. Another entity, such as an insurance provider, may only be interested in data associated with services that are performed as part of routine health care treatment, which may include standard of care services. The unified multiple entity management system may be further configured to divide responsibility for the various health care services among two or more entities. For example, the unified multiple entity management system may be configured to generate invoices assigned to various entities for the health services for which that entity is likely to be responsible for. For example, even though some health care services may be relevant to a trial, the service may also be covered by an insurer under routine health care treatment. Thus, responsibility may be assigned to an insurer for these service(s) instead of a trial sponsor or CRO. Various techniques can be used to divide the responsibility for the various health care services among the different entities including, for example, a manual approach in which input is received selecting a particular entity as being responsible for a particular service and/or automated approaches where rules are defined based on one or more criteria for dividing the responsibility for the services between entities. In some embodiments, artificial intelligence may be used to assist in the prediction of which entity is the best candidate for being responsible for a service.

Thus, in some embodiments, a multiple entity management system may receive information associated with health care services provided to a patient, such as an insurance claim, for example, and associate a first portion of the health care services with a first entity, such as an insurer, and associate a second portion of the health care services with a second entity, such as a CRO or clinical trial sponsor. The multiple entity management system may use, for example, an Artificial Intelligence (AI) based engine to determine the probabilities that the first entity may refuse responsibility for respective ones of the health care services. This determination may be based on, for example, rules defined in a patient's agreement with the first entity (e.g., rules defining a patient's benefit plan), legal regulations (e.g., regulations associated with Medicare or Medicaid), and/or past history of payment by the first entity. The second portion of the health care services that are assigned to the second entity may comprise those health care services whose probabilities exceed a defined threshold. The multiple entity management system may assign responsibility for the first portion of the health care services to the first entity and may assign responsibility for the second portion of the health care services to the second entity. In accordance with some embodiments of the inventive concept, assigning responsibility may include communicating an invoice for the associated health care services to the entity that was assigned responsibility.

The multiple entity management system may also provide a unified reconciliation system that may be used to evaluate payments from both the first and second entities and determine whether they satisfy the invoices in full, in part, or if the invoices have been rejected or declined. A provider may be notified of the status of the invoices, e.g., paid in full, paid in part, pending, or declined, from each of the different entities allowing the provider to follow up on those invoices with unsatisfied portions or rejected entirely to evaluate the reasons why they were not paid. The unified reconciliation system may also facilitate the processing of rejected invoices that may result, for example, from an invoice being assigned incorrectly to an entity. For example, an insurer may be assigned responsibility for an invoice for health care services provided to a patient that were predicted to be part of routine care or standard of care treatment, but this prediction was in error. The unified reconciliation system may be configured to re-direct such an invoice to another entity, such as a CRO or clinical trial sponsor.

Thus, some embodiments of the inventive concept may provide a multiple entity management system that may alleviate the need for a provider to accurately decide which health care services to allocate to a CRO/clinical trial sponsor and which to allocate to an insurer when a patient is participating in a clinical trial as the division of responsibility may be determined using an objective system that is trained in the rules and regulations associated with one or more of the entities, e.g., payors, involved. The improved accuracy may provide benefits to all parties involved: For the provider, the improved accuracy may reduce the risk of penalties or legal liability of improperly billing an insurer for a health care service that should be billed to a CRO or clinical trial sponsor. The improved accuracy may also reduce the number of invoices rejected in whole or in part resulting in a more efficient invoicing process. The provider and the medical system or practice of which the provider is a part may also benefit from the reduced overhead of no longer having to maintain separate independent systems—one for the CRO or clinical trial sponsor and one for an insurer—for managing health care services for patients involved in clinical trials. The unified approach to managing multiple entity responsibility assignment for health care services may reduce computing system costs and management costs associated therewith by avoiding duplicating common processes across multiple systems. Insurers may benefit by receiving invoices that are less likely to be rejected resulting in a more efficient invoicing and remittance process. CROs or clinical trial sponsors may benefit through more accurate billing. Previously, a provider may have assigned responsibility for health care services to a CRO or clinical trial sponsor out of fear of improperly billing an insurer even though those services may be covered by the patient's health care treatment benefit plan with the insurer. The intelligent responsibility assignment functionality provided by the multiple entity management system may allow providers to invoice insurers with greater confidence that the services are covered under health care treatment benefit plans thereby resulting in economic savings for CROs or clinical trial sponsors.

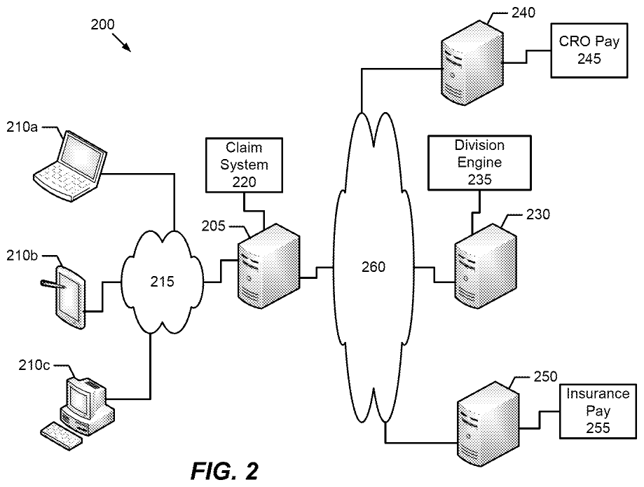

Referring to FIG. 2, a communication network 200 including a multiple entity management system for dividing health care service responsibilities between entities, in accordance with some embodiments of the inventive concept, comprises a health care facility server 205 that is coupled to devices 210a, 210b, and 210c via a network 215. The health care facility may be any type of health care or medical facility, such as a hospital, doctor's office, specialty center (e.g., surgical center, orthopedic center, laboratory center etc.), or the like. The health care facility server 205 may be configured with a claim system module 220 to manage patient files and facilitate the compilation of patient care data. This patient care data may be generated from health care services provided to the patients through health care service providers. The providers may use devices, such as devices 210a, 210b, and 210c, to manage patients' electronic records and to issue orders for the patients. An order may include, but is not limited to, a treatment, a procedure (e.g., surgical procedure, physical therapy procedure, radiologic/imaging procedure, etc.) a test, a prescription, and the like. Thus, the claim system module 220 of the health care facility server 205 may be configured to process the data in the patients' electronic medical records and to compile this information into potential claims for payment from one or more entities on behalf of the individual patients. The network 215 communicatively couples the devices 210a, 210b, and 210c to the health care facility server 205. The network 215 may comprise one or more local or wireless networks to communicate with the health care facility server 205 when the health care facility server 205 is located in or proximate to the health care facility. When the health care facility server 205 is in a remote location from the health care facility, such as part of a cloud computing system or at a central computing center, then the network 215 may include one or more wide area or global networks, such as the Internet.

According to some embodiments of the inventive concept, providers or their medical center or practice may access a multiple entity management server 230 to assist them in dividing the responsibilities for the health care services delivered to patients to one or more entities. For example, as discussed above, when a patient is enrolled in a clinical trial, the responsibility for the health care services may be divided between multiple entities, such as an insurance payor and a CRO or clinical trial sponsor payor. The multiple entity management server 230 may include a division engine module 235 that is configured to receive the information associated with health care services provided to a patient from the health care facility server 205 and associate portions of those health care services with different entities. In the example shown in FIG. 2, two different entities are shown: a CRO or clinical trial sponsor entity is represented by a CRO server 240 and an insurance entity is represented by an insurance server 250. In associating the health care services with the different entities, the division engine module 235 may use an AI engine, for example, to learn what types of health care services are likely to be covered based on health care treatment insurance benefit plans, legal regulations, and/or payment history for the insurer. The division engine 235 may be further configured to assign responsibility for the portions of the health care services assigned to the different entities by, for example, issuing invoices to the different entities for the health care services that they have been assigned. The CRO server 240 may include a CRO pay module 245, which is configured to receive invoices from the multiple entity management server 230, process them, and remit payment in full, remit payment in part, or reject/decline the invoice. In similar fashion, the insurance server 250 may include an insurance pay module 255, which is configured to receive invoices from the multiple entity management server 230, process them, and remit payment in full, remit payment in part, or reject/decline the invoice. The division engine module 235 may further comprise a reconciliation capability to evaluate payments from both the CRO server 240 and the insurance server 250 and determine whether they satisfy the invoices in full, in part, or if the invoices have been rejected or declined. The division engine module 235 may be configured to notify a provider of the status of the invoices, e.g., paid in full, paid in part, pending, or declined, from each of the different entities to allow the provider to follow up on those invoices to correct any defects that are preventing their payment.

Although only two entities—a CRO and an insurer—are illustrated in FIG. 2, it will be understood that embodiments of the inventive concept are not limited to two entities and the multiple entity management server 230 may be configured to divide health care service responsibilities between more than two entities in accordance with other embodiments of the inventive concept.

Although FIG. 2 illustrates an example communication network including a multiple entity manager system for dividing health care service responsibilities between entities, it will be understood that embodiments of the inventive subject matter are not limited to such configurations, but are intended to encompass any configuration capable of carrying out the operations described herein.

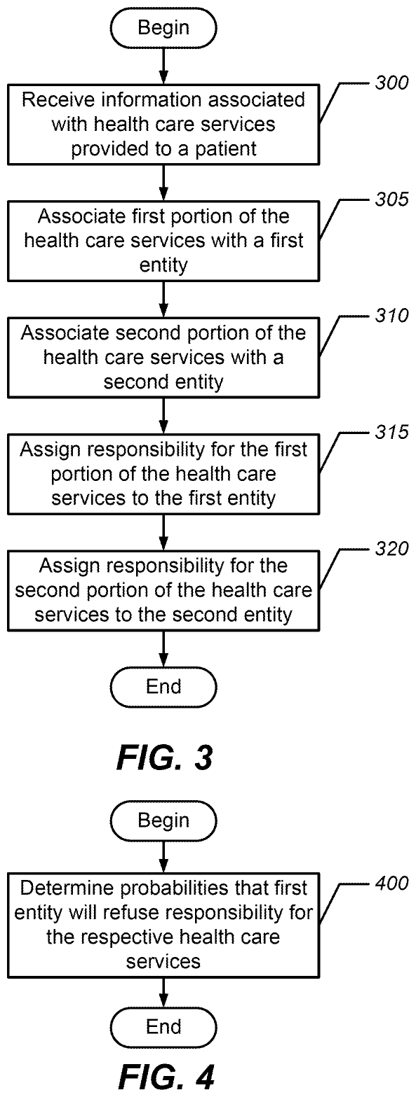

FIGS. 3-8 are flowcharts that that illustrate operations for dividing health care service responsibilities between entities in accordance with some embodiments of the inventive concept. Referring now to FIG. 3, the multiple entity management server 230 may receive information associated with health care services provided to a patient from the health care facility server 205 (Block 300). The division engine 235 may associate a first portion of the health services with a first entity (Block 305) (e.g., insurance payor) and associate a second portion of the health care services with a second entity (Block 310) (e.g., CRO payor). The division engine 235 may be further configured to assign responsibility for the first portion of the health care services to the first entity (Block 315) and to assign responsibility for the second portion of the health care services to the second entity (Block 320).

Referring now to FIG. 4, in some embodiments, the division engine may use, for example, an AI engine to determine probabilities that the first entity will refuse responsibility for respective ones of the health care services (Block 400). In some embodiments, this determination may be based on rules defined in a patient's agreement with the first entity (e.g., rules defining a patient's benefit plan), legal regulations (e.g., regulations associated with Medicare or Medicaid), and/or past history of payment by the first entity. In this regard, the AI system may learn over time the behavior of one or more of the entities and may use this knowledge in more accurately assigning responsibility for the various health care services consumed by a patient. The second portion of the health care services that are assigned to the second entity may comprise those health care services whose probabilities exceed a defined threshold. In some embodiments where only two entities are involved, responsibility for all health care services not assigned to a first entity may be assigned to the second entity. When more than two entities are involved, responsibility all health care services not assigned to the first entity may be distributed among the remaining entities. The multiple entity management server 230 and division engine 235 may provide a unified system that provides each entity with the health care service data in a recognizable format to allow these entities to accept responsibility through, for example, processing and payment of invoice(s) for the service(s).

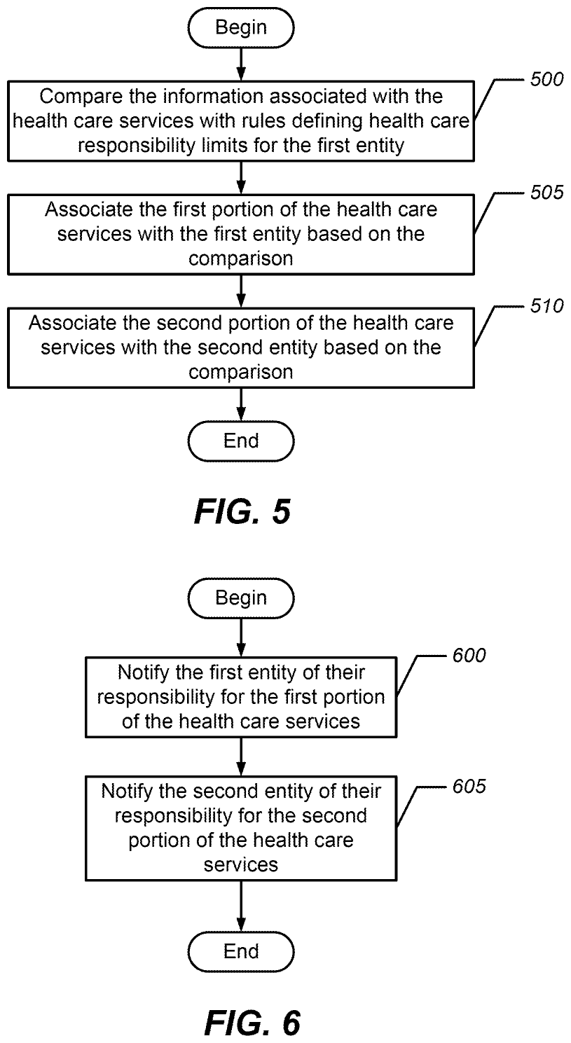

Referring now to FIG. 5, specific embodiments for determining the probabilities that the first entity will refuse responsibility for the respective ones of the health care services will now be described. Various factors may influence the likelihood that the first entity will refuse responsibility for one or more health care services including, but not limited to, whether the first entity has an agreement with a patient in the form of an insurance contract, which may define rules establishing what health care services the first entity has agreed to reimburse, whether there are any applicable legal regulations (e.g., Medicare or Medicaid regulations), and/or payment history information associated with the first entity where payment for various services has been previously approved, denied, and/or negotiated. Thus, the division engine 235 may compare information associated with the health care services with the rules defining health care responsibility limits for the first entity (Block 500). The division engine 235 may associate the first portion of the health care services with the first entity based on the comparison (Block 505) and may associate the second portion of the health care services with the second entity based on the comparison (Block 510).

Referring now to FIG. 6, the division engine 235 may be further configured to notify the first entity of their responsibility for the first portion of the health care services (Block 500) and may notify the second entity of their responsibility for the second portion of the health care services (Block 505). These notifications may comprise, for example, one or more invoices demanding payment for health care services identified thereon.

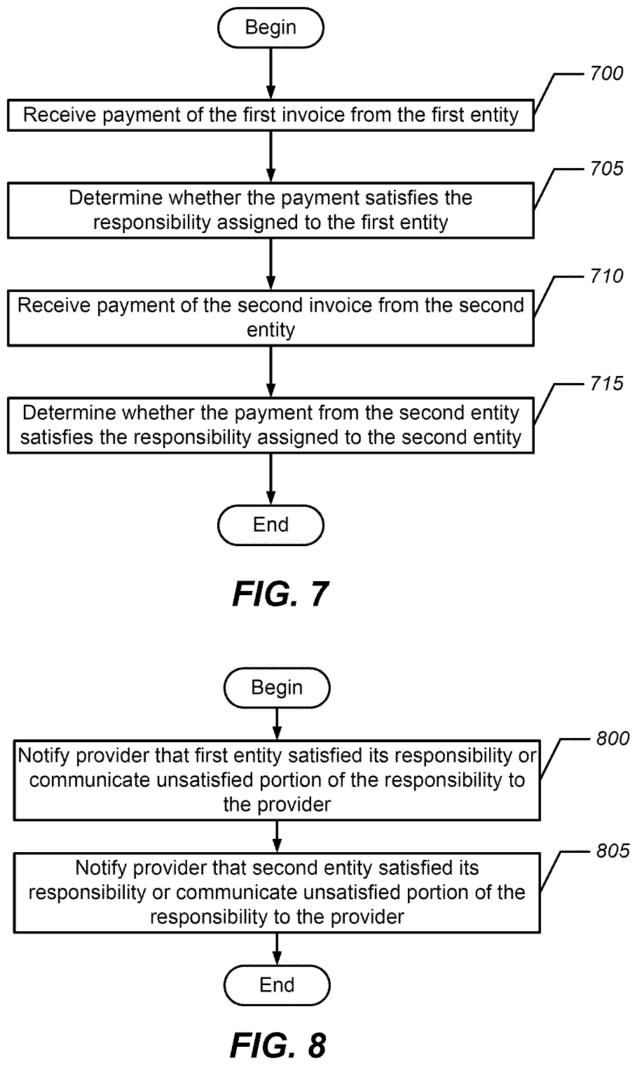

As described above, the division engine 235 may provide a unified reconciliation capability for reconciling payment of invoices, for example, from multiple entities, such as a CRO or clinical trial sponsor or an insurer. Referring now to FIG. 7, the multiple entity management server 230 may receive payment of a first invoice from the first entity (Block 700). The division engine 235 may determine whether the payment satisfies the responsibility assigned to the first entity (Block 705). In some embodiments, the division engine 235 may determine whether the invoice was paid in full, paid in part, or rejected/declined. The division engine 235 may also keep track of pending invoices for which payment has not been received from the first entity. Similarly, the multiple entity management server 230 may receive payment of a second invoice from the second entity (Block 710). The division engine 235 may determine whether the payment satisfies the responsibility assigned to the second entity (Block 715). Similar to the first entity, the division engine 235 may determine whether the invoice was paid in full, paid in part, or rejected/declined and may also keep track of pending invoices for which payment has not been received from the second entity.

Referring now to FIG. 8, the division manager 235 may notify the provider that the first entity has satisfied its responsibility for the health care services for which it was assigned through, for example, payment of one or more invoices. Any unsatisfied portion of the first entity's responsibility, e.g., invoices that are unpaid in whole or in part, may be communicated to the provider through the health care facility server 205 to allow the provider to further evaluate and correct any errors that are preventing, for example, an invoice from being paid by the first entity (Block 800). Similarly, the division manager 235 may notify the provider that the second entity has satisfied its responsibility for the health care services for which it was assigned through, for example, payment of one or more invoices. Any unsatisfied portion of the second entity's responsibility, e.g., invoices that are unpaid in whole or in part, may be communicated to the provider through the health care facility server 205 to allow the provider to further evaluate and correct any errors that are preventing, for example, an invoice from being paid by the second entity (Block 805). In some embodiments, invoices that are unpaid in whole or in part may be directly communicated to another entity for processing. For example, if an invoice was rejected in whole or in part by an insurer, the invoice may automatically be forwarded to a trial sponsor or CRO for payment to account for the deficiency.

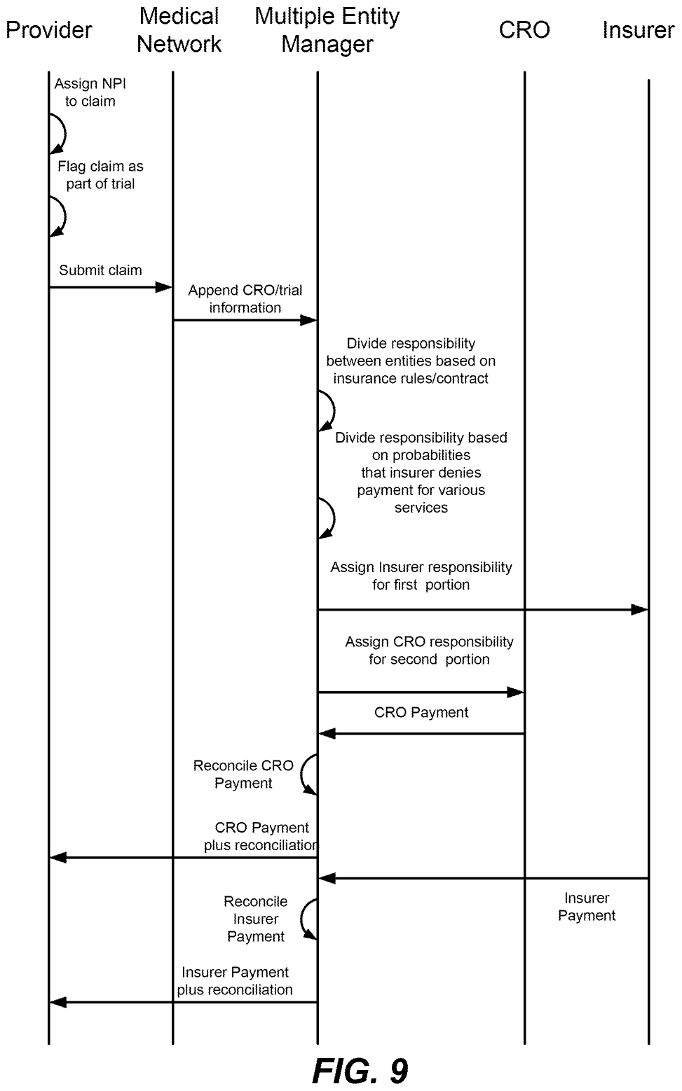

FIG. 9 is a message flow diagram that illustrates operations for dividing health care service responsibilities between entities in accordance with some embodiments of the inventive concept. In the example of FIG. 9, the different entities comprise a CRO and an insurer, but it will be understood that additional or different entities may be used in accordance with other embodiments of the inventive concept. Referring now to FIG. 9, the message flow diagram includes five actors: a provider, a medical network, a multiple entity manager, a CRO, and an insurer. As described above, a provider's health care facility server 205 may be configured to compile information associated with health care services provided to a patient into a claim for payment. Providers are assigned individual codes known as National Provider Identifier (NPI) codes. The provider's NPI is assigned to the claim and the claim may be flagged as being part of a trial, i.e., the claim has multiple entities (CRO and insurer) that are responsible for the different portions of the health care services provided to the patient. When the medical facility or practice of which the provider is a part uses a third-party billing system, then the claim is forwarded to a medical network, which appends CRO/trial information to the claim before forwarding it to the multiple entity manager. As described above with respect to the multiple entity management server 230 and division engine module 235, the multiple entity manager may divide responsibility between the CRO and the insurer based on insurance or legal rules, and/or a contract between the insurer and the patient. An AI system may, in some embodiments, be configured to use the legal rules, insurance rules, contract provisions, and learned past history of payment behavior of the insurer to divide the responsibilities between the insurer and the CRO for the health care services provided to the patient based on probabilities that the insurer denies or rejects payment for the respective ones of the health care services. The multiple entity manager may then assign the insurer responsibility for a first portion of the health care services and may assign the CRO responsibility for a second portion of the health care services. Specifically, one or more invoices for the first portion of the health care services may be sent to the insurer and one or more invoices for the second portion of the health care services may be sent to the CRO. The multiple entity manager may receive payment from the CRO and may reconcile the payment to determine if the CRO has paid the invoices in full, in part, or has rejected all of the invoice(s). The CRO payment may then be forwarded to the provider along with the reconciliation information allowing the provider to resubmit any partially or fully rejected invoices. Similarly, the multiple entity manager may receive payment from the insurer and may reconcile the payment to determine if the insurer has paid the invoices in full, in part, or has rejected all of the invoice(s). The insurer payment may then be forwarded to the provider along with the reconciliation information allowing the provider to resubmit any partially or fully rejected invoices.

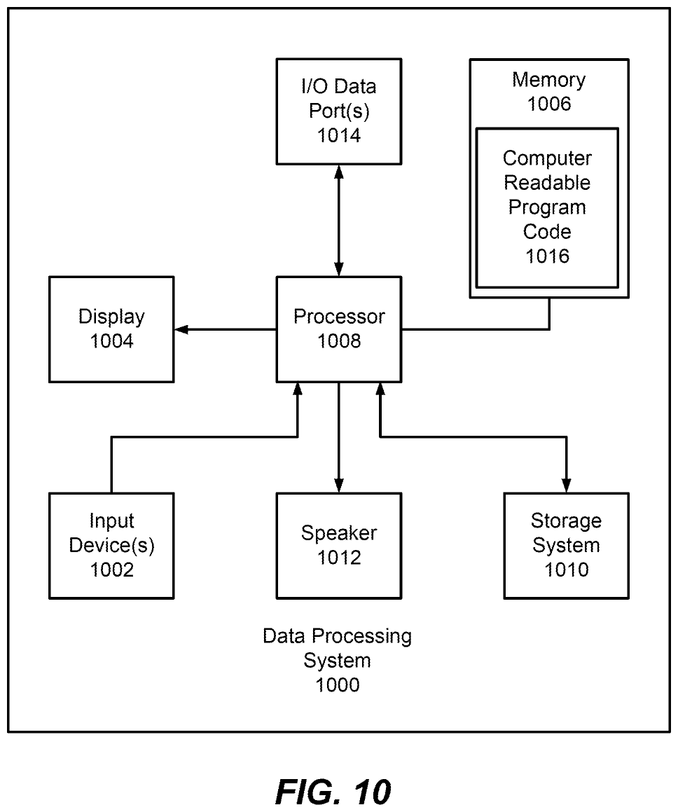

Referring now to FIG. 10, a data processing system 1000 that may be used to implement the multiple entity management server 230 of FIG. 2, in accordance with some embodiments of the inventive concept, comprises input device(s) 1002, such as a keyboard or keypad, a display 1004, and a memory 1006 that communicate with a processor 1008. The data processing system 1000 may further include a storage system 1010, a speaker 1012, and an input/output (I/O) data port(s) 1014 that also communicate with the processor 1008. The processor 1008 may be, for example, a commercially available or custom microprocessor. The storage system 1010 may include removable and/or fixed media, such as floppy disks, ZIP drives, hard disks, or the like, as well as virtual storage, such as a RAMDISK. The I/O data port(s) 1014 may be used to transfer information between the data processing system 1000 and another computer system or a network (e.g., the Internet). These components may be conventional components, such as those used in many conventional computing devices, and their functionality, with respect to conventional operations, is generally known to those skilled in the art. The memory 1006 may be configured with computer readable program code 1016 to facilitate division of health care service responsibilities between entities according to some embodiments of the inventive concept.

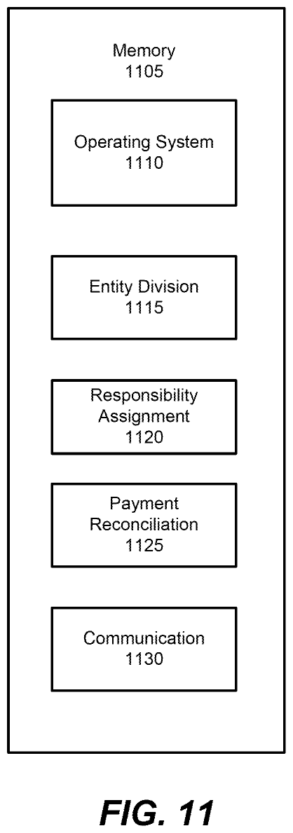

FIG. 11 illustrates a memory 1105 that may be used in embodiments of data processing systems, such as the multiple entity management server 230 of FIG. 2 and the data processing system 1000 of FIG. 10, respectively, to facilitate division of health care service responsibilities between entities according to some embodiments of the inventive concept. The memory 1105 is representative of the one or more memory devices containing the software and data used for facilitating operations of the multiple entity management server 230 and division engine 235 as described herein. The memory 1105 may include, but is not limited to, the following types of devices: cache, ROM, PROM, EPROM, EEPROM, flash, SRAM, and DRAM. As shown in FIG. 11, the memory 1105 may contain five or more categories of software and/or data: an operating system 1110, an entity division module 1115, a responsibility assignment module 1120, a payment reconciliation module 1125, and a communication module 1130. In particular, the operating system 1110 may manage the data processing system's software and/or hardware resources and may coordinate execution of programs by the processor. The entity division module 1115, the responsibility assignment module 1120, the payment reconciliation module 1125, and the communication module 1130 may be configured to perform one or more operations described above with respect to the multiple entity management server 230. In some embodiments, the entity division module 1115 may be configured to perform one or more of the operations described above with respect to blocks 305 and 310 of FIG. 3, block 400 of FIG. 4, and blocks 500, 505, and 510 of FIG. 5. The responsibility assignment module 1120 may be configured to perform one or more operations described above with respect to blocks 315 and 320 of FIG. 3. The payment reconciliation module 1125 may be configured to perform one or more operations described above with respect to blocks 700, 705, 710, and 715 of FIG. 7 and blocks 800 and 805 of FIG. 8. The communication module 1130 may be configured to perform one or more operations described above with respect to blocks 600 and 605 of FIG. 6 and blocks 800 and 805 of FIG. 8.

Although FIGS. 10-11 illustrate hardware/software architectures that may be used in data processing systems, such as the multiple entity management server 230 of FIG. 2 and the data processing system 1000 of FIG. 10, respectively, in accordance with some embodiments of the inventive concept, it will be understood that the present invention is not limited to such a configuration but is intended to encompass any configuration capable of carrying out operations described herein.

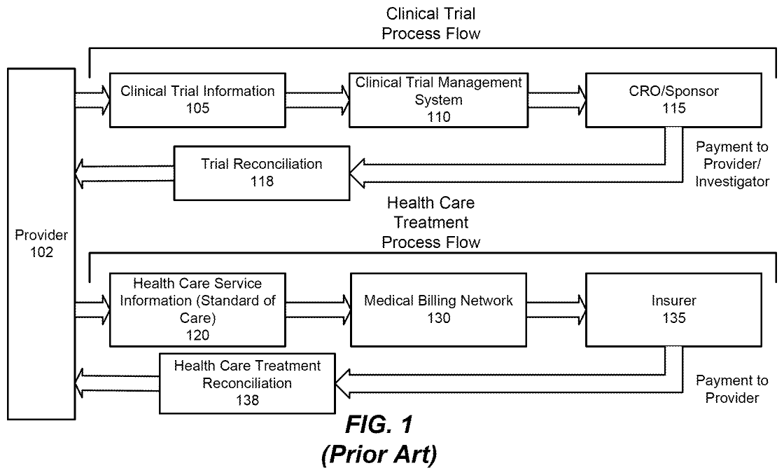

Computer program code for carrying out operations of data processing systems discussed above with respect to FIGS. 1-11 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the multiple entity management server 230 of FIG. 2 and the data processing system 1000 of FIG. 10 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 2-11 may be used to facilitate division of health care service responsibilities according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1105 when coupled to a processor includes computer readable program code that, when executed by the processor, causes the processor to perform operations including one or more of the operations described herein with respect to FIGS. 2-9.

Some embodiments of the inventive concept may provide a multiple entity management system for dividing responsibility for health care services provided to a patient among multiple entities. This may be useful, for example, in managing responsibility for patient care when the patient is enrolled in a clinical trial and a CRO and an insurer share responsibility for reimbursing the provider for health care services that have been provided. The multiple entity management system may replace the dual, parallel systems that are frequently used to manage patient care during a clinical trial with a single consolidated management system that relieves the provider of the burden of managing two different systems and provides improved accuracy in dividing responsibility, e.g., dividing invoices, between the CRO and the insurer. The improved accuracy may also reduce the potential for penalties or other legal liability due to overbilling insurance payors, such as government insurance providers (e.g., Medicare and Medicaid), with invoices that should have been assigned to the CRO.

### FURTHER DEFINITIONS AND EMBODIMENTS

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
receiving information associated with health care services provided to a patient;
determining for respective ones of the health care services respective probabilities that a first entity will refuse responsibility;
associating a first portion of the health care services with the first entity;
associating a second portion of the health care services with a second entity, the second portion of the health care services comprising respective ones of the health care services having probabilities that exceed a defined threshold;
assigning responsibility for the first portion of the health care services to the first entity; and
assigning responsibility for the second portion of the health care services to the second entity.

2. The method of claim 1, further comprising:
determining that multiple entities are responsible for the health care services provided to the patient based on the information that was received;
wherein associating the first portion of the health care services with the first entity comprises associating the first portion of the health care services with the first entity responsive to determining that multiple entities are responsible for the health care services; and
wherein associating the second portion of the health care services with the second entity comprises associating the second portion of the health care services with the second entity responsive to determining that multiple entities are responsible for the health care services.

3. The method of claim 1, wherein assigning responsibility for the first portion of the health care services to the first entity comprises:
notifying the first entity of their responsibility for the first portion of the health care services; and
wherein assigning responsibility for the second portion of the health care services to the second entity comprises:
notifying the second entity of their responsibility for the second portion of the health care services.

4. The method of claim 3, wherein notifying the first entity of their responsibility for the first portion of the health care services comprises:
communicating a first invoice for the first portion of the health care services to the first entity; and
wherein notifying the second entity of their responsibility for the second portion of the health care services comprises:
communicating a second invoice for the second portion of the health care services to the second entity.

5. The method of claim 4, further comprising:
receiving payment of the first invoice from the first entity;
determining whether the payment from the first entity satisfies the responsibility for the first portion of the health care services assigned to the first entity;
receiving payment of the second invoice from the second entity; and
determining whether the payment from the second entity satisfies the responsibility for the second portion of the health care services assigned to the second entity.

6. The method of claim 5, further comprising:
determining a first unsatisfied portion of the responsibility for the first portion of the health care services assigned to the first entity when the payment from the first entity does not satisfy the responsibility for the first portion of the health care services assigned to the first entity; and
determining a second unsatisfied portion of the responsibility for the second portion of the health care services assigned to the second entity when the payment from the second entity does not satisfy the responsibility for the second portion of the health care services assigned to the second entity.

7. The method of claim 6, further comprising:
communicating the first unsatisfied portion of the responsibility for the first portion of the health care services assigned to the first entity to a provider of the health care services and/or the second entity; and
communicating the second unsatisfied portion of the responsibility for the second portion of the health care services assigned to the second entity to the provider of the health care services.

8. The method of claim 5, further comprising:
notifying a provider that the payment from the first entity satisfies the responsibility for the first portion of the health care services assigned to the first entity when the payment from the first entity does satisfy the responsibility for the first portion of the health care services assigned to the first entity; and
notifying the provider that the payment from the second entity satisfies the responsibility for the second portion of the health care services assigned to the second entity when the payment from the second entity does satisfy the responsibility for the second portion of the health care services assigned to the second entity.

9. The method of claim 1, further comprising:
comparing the information associated with the health care services with rules defining health care service responsibility limits for the first entity;
wherein associating the first portion of the health care services with the first entity comprises:
associating the first portion of the health care services with the first entity based on comparing the information associated with the health care services with the rules defining the health care service responsibility limits for the first entity; and
wherein associating the second portion of the health care services with the second entity comprises:
associating the second portion of the health care services with the second entity based on comparing the information associated with the health care services with the rules defining the health care service responsibility limits for the first entity.

10. The method of claim 1, wherein the first entity is an insurer; and
wherein the second entity is a contract research organization.

11. The method of claim 10, wherein the first portion of the health care services are associated with health care treatment services; and
wherein the second portion of the health care services are associated with a clinical trial.

12. A system, comprising:
a processor; and
a memory coupled to the processor and comprising computer readable program code embodied in the memory that is executable by the processor to perform operations comprising:
receiving information associated with health care services provided to a patient;
determining for respective ones of the health care services respective probabilities that a first entity will refuse responsibility;
associating a first portion of the health care services with the first entity;
associating a second portion of the health care services with a second entity, the second portion of the health care services comprising respective ones of the health care services having probabilities that exceed a defined threshold;
assigning responsibility for the first portion of the health care services to the first entity; and
assigning responsibility for the second portion of the health care services to the second entity.

13. The system of claim 12, wherein assigning responsibility for the first portion of the health care services to the first entity comprises:
notifying the first entity of their responsibility for the first portion of the health care services; and
wherein assigning responsibility for the second portion of the health care services to the second entity comprises:
notifying the second entity of their responsibility for the second portion of the health care services.

14. The system of claim 12, wherein the operations further comprise:
comparing the information associated with the health care services with rules defining health care service responsibility limits for the first entity;
wherein associating the first portion of the health care services with the first entity comprises:
associating the first portion of the health care services with the first entity based on comparing the information associated with the health care services with the rules defining the health care service responsibility limits for the first entity; and
wherein associating the second portion of the health care services with the second entity comprises:
associating the second portion of the health care services with the second entity based on comparing the information associated with the health care services with the rules defining the health care service responsibility limits for the first entity.

15. The system of claim 12, wherein the first entity is an insurer; and
wherein the second entity is a contract research organization.

16. The method of claim 15, wherein the first portion of the health care services are associated with health care treatment services; and
wherein the second portion of the health care services are associated with a clinical trial.

17. A computer program product, comprising:
a tangible computer readable storage medium comprising computer readable program code embodied in the medium that is executable by a processor to perform operations comprising:
receiving information associated with health care services provided to a patient;
determining for respective ones of the health care services respective probabilities that a first entity will refuse responsibility;
associating a first portion of the health care services with the first entity;
associating a second portion of the health care services with a second entity, the second portion of the health care services comprising respective ones of the health care services having probabilities that exceed a defined threshold;
assigning responsibility for the first portion of the health care services to the first entity; and
assigning responsibility for the second portion of the health care services to the second entity.

18. The computer program product of claim 17, wherein assigning responsibility for the first portion of the health care services to the first entity comprises:
notifying the first entity of their responsibility for the first portion of the health care services; and
wherein assigning responsibility for the second portion of the health care services to the second entity comprises:
notifying the second entity of their responsibility for the second portion of the health care services.

19. The computer program product of claim 17, wherein the operations further comprise:
comparing the information associated with the health care services with rules defining health care service responsibility limits for the first entity;
wherein associating the first portion of the health care services with the first entity comprises:
associating the first portion of the health care services with the first entity based on comparing the information associated with the health care services with the rules defining the health care service responsibility limits for the first entity; and
wherein associating the second portion of the health care services with the second entity comprises:
associating the second portion of the health care services with the second entity based on comparing the information associated with the health care services with the rules defining the health care service responsibility limits for the first entity.

20. The computer program product of claim 17, wherein the first entity is an insurer;
wherein the second entity is a contract research organization;
wherein the first portion of the health care services are associated with health care treatment services; and
wherein the second portion of the health care services are associated with a clinical trial.
